(19) United States
(12) Patent Application Publication
(10) Pub. No.: US 2017/0114356 A1
(43) Pub. Date: Apr. 27, 2017

(54) NOVEL ALTERNATIVELY SPLICED TRANSCRIPTS AND USES THEREOF FOR IMPROVEMENT OF AGRONOMIC CHARACTERISTICS IN CROP PLANTS

(71) Applicant: E I DU PONT DE NEMOURS AND COMPANY, Wilmington, DE (US)

(72) Inventors: BAILIN LI, HOCKESSIN, DE (US); SHAWN THATCHER, NEWARK, DE (US)

(21) Appl. No.: 15/047,804

(22) Filed: Feb. 19, 2016

Related U.S. Application Data
(60) Provisional application No. 62/257,774, filed on Nov. 20, 2015.
(60) Provisional application No. 62/118,576, filed on Feb. 20, 2015.

(51) Int. Cl. C12N 15/82; C07K 14/415; C12Q 1/68

(52) U.S. Cl. CPC C12N 15/8261; C12Q 1/6895; C07K 14/415; C12Y 301/00; C12Q 2600/158; C12Q 2600/13; C12Q 2600/156

(57) ABSTRACT

Computational analysis of hundreds of RNA-seq libraries enabled the identification of novel transcripts in maize. The novel transcripts are provided herein, as are recombinant DNA constructs comprising such, transgenic plants or cell thereof comprising the recombinant DNA constructs, and methods for generating transgenic seed and plants with improved agronomic characteristics.

### CROSS REFERENCE TO RELATED APPLICATIONS

This application claims the benefit of U.S. Provisional Application No. 62/118,576, filed Feb. 20, 2015, and U.S. Provisional Application No. 62/257,774, filed Nov. 20, 2015, the entire contents of which are herein incorporated by reference.

### INCORPORATION OF SEQUENCE LISTING

Two copies of the sequence listing (Seq. Listing Copy 1 and Seq. Listing Copy 2) and a computer-readable form of the sequence listing, all on CD-ROMs, each containing the file named
20160217_BB2527USNP_SequenceListing_ST25.txt, which is 592,118 kbs (measured in MS-DOS) and was created on Feb. 17, 2016, are herein incorporated by reference.

### FIELD

The field relates to plant breeding and genetics and, in particular, to recombinant DNA constructs useful for production of transgenic plants with improved agronomic characteristics.

### BACKGROUND

The ability to develop transgenic plants with improved agronomic characteristics depends in part on the identification of genes that are useful for production of transformed plants for expression of novel polypeptides.

### SUMMARY

Novel polynucleotides identified in maize and the polypeptides encoded by such are provided herein. The polynucleotide sequences are represented by SEQ ID NOs:1-157,066 and 198,539-222,468. Novel polypeptides encoded by polynucleotides disclosed herein are represented by SEQ ID NOs:157,067-198,538 and 222,469-228,453. The polynucleotides are useful for improvement of one or more agronomic characteristics in crop plants.

Recombinant DNA constructs comprising the polynucleotides disclosed herein are also provided. A recombinant DNA construct may comprise a polynucleotide operably linked to at least one regulatory sequence wherein said polynucleotide comprises (a) a nucleic acid sequence of at least 95% sequence identity, based on the Clustal V method of alignment, when compared to any of SEQ ID NOs:1-157,066 and 198,539-222,468; (b) a nucleic acid sequence encoding an amino acid sequence of at least 95% sequence identity, based on the Clustal V method of alignment, when compared to any of SEQ ID NOs:157,067-198,538 and 222,469-228,453; or (c) a nucleic acid sequence that is transcribed into an RNA molecule that suppresses the level of an endogenous polypeptide having an amino acid sequence of at least 95% sequence identity, based on the Clustal V method of alignment, when compared to any of SEQ ID NOs:157,067-198,538 and 222,469-228,453. The regulatory sequence may be a promoter functional in a plant cell.

Such constructs are useful for production of transgenic plants having one or more improved agronomic characteristics as the result of increased or decreased expression of a polypeptide disclosed herein.

Methods for producing a transgenic plant with an improved agronomic characteristic are provided in which a plant cell is transformed with a recombinant DNA construct disclosed herein and a plant is regenerated from the transformed plant cell.

Transgenic plant cells, transgenic plants comprising the plant cells, and seed produced from the transgenic plants, e.g. transgenic crop plants such as maize, soybean, sunflower, sorghum, canola, wheat, alfalfa, cotton, rice, barley, millet, sugar cane, and switchgrass, which comprise a recombinant DNA construct disclosed herein, are also provided.

Methods for introducing any of the polynucleotides disclosed herein into a target site in the genome of a plant cell are also provided. The methods comprise (a) introducing into a plant cell one recombinant DNA construct capable of expressing a guide RNA and another recombinant DNA construct capable of expressing a Cas endonuclease, wherein said guide RNA and Cas endonuclease are capable of forming a complex that enables the Cas endonuclease to introduce a double strand break at said target site; (b) contacting the plant cell with a donor DNA comprising a polynucleotide of interest, wherein said polynucleotide of interest is any of the polynucleotides disclosed herein; and (c) identifying at least one plant cell that has the polynucleotide of interest integrated into the target site. The polynucleotide of interest may be a nucleic acid sequence of at least 95% sequence identity, based on the Clustal V method of alignment, when compared to any of SEQ ID NOs:1-157,066 and 198,539-222,468; or a nucleic acid sequence encoding an amino acid sequence of at least 95% sequence identity, based on the Clustal V method of alignment, when compared to any of SEQ ID NOs:157,067-198,538 and 222,469-228,453.

Methods of marker assisted selection of a maize plant are also provided in which the methods include: analyzing for expression of one or more transcripts selected from a group consisting of nucleotide sequences, wherein the nucleotide sequences encode alternatively spliced isoforms; correlating one or more transcripts with an improved agronomic characteristic; and selecting for the improved agronomic characteristic in a maize plant by assaying one or more markers that detect the one or more transcripts associated with the improved agronomic characteristic. The expression analysis may be performed with a plurality of isoform-specific probes derived from the group consisting of sequences SEQ ID NOs:1-157,066 and 198,539-222,468.

Methods for enhancing expression of a transgene in a plant are provided in which a nucleotide sequence of a transgene or an amino acid sequence of a transgene are obtained; the sequences are compared to a collection of nucleotide sequences of alternatively spliced isoforms or to a collection of amino acid sequences encoded by the alternatively spliced isoforms; one or more alternatively spliced isoform sequences corresponding to a transgene are selected; and the one or more alternatively spliced isoform sequences in the plant are expressed, thereby enhancing expression of the transgene. The selected isoform sequence may be expressed under its native promoter or a constitutive or tissue-preferred promoter.

Methods of identifying alternatively spliced isoforms of one or more genes involved in an agronomic trait are also provided in which a plurality of transcripts that are expressed under an abiotic stress condition are sequenced and the sequenced transcripts are compared to transcript sequences that are expressed in a non-stressed condition. Genes with splicing patterns that differ between the abiotic stress condition and non-stressed condition are then detected.

Methods of increasing yield in a plant are provided in which a spliced isoform is expressed or its expression is reduced, wherein the nucleotide for expression or a silencing element to reduce the expression of the spliced isoform is derived from a sequence selected from the group consisting of SEQ ID NOs: 1-157,066 and 198,539-222,468. The plant may be maize.

Methods of genome editing are provided in which one or more heterologous splice sites are introduced into one or more genomic loci of a plant, or one or more splice sites of the plant are selectively eliminated. The methods include identifying one or more alternatively spliced isoforms; determining one or more splice sites in the genomic region for the alternatively spliced isoforms; and introducing a splice site in the genomic loci that lacks the one or more splice sites or changing one or more nucleotides in a preexisting splice site to render the preexisting splice site non-functional. The alternatively spliced isoforms may be selected from the group consisting of SEQ ID NOs: 1-157,066 and 198,539-222,468.

Computer systems comprising: a relational database having records containing a) information about one or more sequences of spliced isoforms represented by SEQ ID NOs: 1-157,066 and 198,539-222,468 or amino acid sequences of 157,067-198,538 and 222,469-228,453; b) information identifying known SNPs or QTLs known to be associated with one or more traits of interest; and c) a user interface allowing a user to access the information contained in the records, are also provided.

Computer programs comprising: a computer-usable medium having computer-readable program code embodied thereon relating to generating a relational database having records containing a) information about one or more sequences of spliced isoforms represented by SEQ ID NOS: 1-157,066 and 198,539-222,468 or amino acid sequences of 157,067-198,538 and 222,469-228,453; b) information identifying known SNPs or QTLs known to be associated with one or more traits of interest; and c) a user interface allowing a user to access the information contained in the records, are also provided.

Methods for comparing a plurality of spliced isoforms among two or more plant populations, comprising: (a) accessing, by a computer system, a database of genetic information comprising spliced isoform sequences obtained from a plurality of plant tissues; (b) categorizing, by a computer system, the data in the database into a plurality of groups of spliced isoforms, such that one or more spliced isoforms for a particular gene are in the same group, and each group represents a different set of spliced isoforms; and (c) inputting data into a computer system, the data comprising sequences of one or more transcripts obtained from the two or more plant populations, are also provided. The plant populations may comprise inbred populations. The database may further comprise QTL information associated with one or more spliced isoforms.

Nucleotide constructs that express one or more guide RNAs, wherein a guide RNA targets a genomic sequence that encodes a polypeptide selected the group consisting of amino acid sequences of SEQ ID NOs: 157,067-198,538 and 222,469-228,453, are also provided.

### BRIEF DESCRIPTION OF THE SEQUENCE LISTING

The disclosure can be more fully understood from the following detailed description and the accompanying Sequence Listing which forms a part of this application.

SEQ ID NOs:1-157,066 and 198,539-222,468 are the cDNA sequences corresponding to the transcripts identified herein. SEQ ID NOs:157,067-198,538 and 222,469-228,453 are the amino acid sequences of polypeptides encoded by polynucleotides disclosed herein. Table 3 provides the isoform identifier associated with each SEQ ID NO:.

The sequence descriptions and Sequence Listing attached hereto comply with the rules governing nucleotide and/or amino acid sequence disclosures in patent applications as set forth in 37 C.F.R. §1.821 1.825.

The Sequence Listing contains the one letter code for nucleotide sequence characters and the three letter codes for amino acids as defined in conformity with the IUPAC IUBMB standards described in Nucleic Acids Res. 13:3021 3030 (1985) and in the Biochemical J. 219 (No. 2):345 373 (1984) which are herein incorporated by reference. The symbols and format used for nucleotide and amino acid sequence data comply with the rules set forth in 37 C.F.R. §1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The terms “monocot” and “monocotyledonous plant” are used interchangeably herein. A monocot as used herein includes the Gramineae.

The terms “dicot” and “dicotyledonous plant” are used interchangeably herein. A dicot as used herein includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

A “trait” refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency (such as for example nitrogen deficiency), nutrient excess, UV irradiation, atmospheric pollution (e.g., ozone) and exposure to chemicals (e.g., paraquat) that induce production of reactive oxygen species (ROS).

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is substantially modified from its native form in composition and/or genomic locus by deliberate human intervention. “Polynucleotide”, “nucleic acid sequence”, “nucleotide sequence”, or “nucleic acid fragment” are used interchangeably and is a polymer of RNA or DNA that is single or double-stranded, optionally containing synthetic, non-natural or altered nucleotide bases. Nucleotides (usually found in their 5′ monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Coding region” refers to the portion of a messenger RNA (or the corresponding portion of another nucleic acid molecule such as a DNA molecule) which encodes a protein or polypeptide. “Non-coding region” refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected m RNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

As used herein, the terms “target site”, “target sequence”, “genomic target site” and “genomic target sequence” are used interchangeably herein and refer to a polynucleotide sequence in the genome of a plant cell or yeast cell that comprises a recognition site for a double-strand-break-inducing agent.

An “endonuclease” refers to an enzyme that cleaves the phosphodiester bond within a polynucleotide chain.

Endonucleases include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex.

Type I and Type III restriction endonucleases recognize specific recognition sites, but typically cleave at a variable position from the recognition site, which can be hundreds of base pairs away from the recognition site. In Type II systems the restriction activity is independent of any methylase activity, and cleavage typically occurs at specific sites within or near to the recognition site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

A “meganuclease” refers to a homing endonuclease, which like restriction endonucleases, bind and cut at a specific recognition site, however the recognition sites for meganucleases are typically longer, about 18 by or more. In some embodiments of the invention, the meganuclease has been engineered (or modified) to cut a specific endogenous recognition sequence, wherein the endogenous target sequence prior to being cut by the engineered double-strand-break-inducing agent was not a sequence that would have been recognized by a native (non-engineered or non-modified) endonuclease.

A “meganuclease polypeptide” refers to a polypeptide having meganuclease activity and thus capable of producing a double-strand break in the recognition sequence.

Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F- , I- , or PI- for enzymes encoded by free-standing open reading frames, introns, and inteins, respectively. For example, intron- , intein- , and freestanding gene encoded meganuclease from Saccharomyces cerevisiae are denoted I-SceI, PI-SceI, and F-SceII, respectively. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764. In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used herein, including, but not limited to, I-SceI, I-SceII, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-AniI, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

TAL effector nucleases are a new class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkq704; and Miller et al. (2011) Nature Biotechnology 29:143-148; all of which are herein incorporated by reference.

As used herein, the term “Cas gene” refers to a gene that is generally coupled, associated or close to or in the vicinity of flanking CRISPR loci.

CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bps, repeated from 1 to 140 times-also referred to as CRISPR-repeats) which are partially palindromic. The repeated sequences (usually specific to a species) are interspaced by variable sequences of constant length (typically 20 to 58 by depending on the CRISPR locus (WO2007/024097published Mar. 1, 2007).

CRISPR loci were first recognized in E. coli (Ishino et al. (1987) J. Bacterial. 169:5429-5433; Nakata et al. (1989) J. Bacterial. 171:3553-3556). Similar interspersed short sequence repeats have been identified in Haloferax mediterranei, Streptococcus pyogenes, Anabaena, and Mycobacterium tuberculosis (Groenen et al. (1993) Mol. Microbiol. 10:1057-1065; Hoe et al. (1999) Emerg. Infect. Dis. 5:254-263; Masepohl et al. (1996) Biochim. Biophys. Acta 1307:26-30; Mojica et al. (1995) Mol. Microbiol. 17:85-93). The CRISPR loci differ from other SSRs by the structure of the repeats, which have been termed short regularly spaced repeats (SRSRs) (Janssen et al. (2002) OMICS J. Integ. Biol. 6:23-33; Mojica et al. (2000) Mol. Microbiol. 36:244-246). The repeats are short elements that occur in clusters, that are always regularly spaced by variable sequences of constant length (Mojica et al. (2000) Mol. Microbiol. 36:244-246). \

The terms “Cas gene”, “CRISPR-associated (Cas) gene” are used interchangeably herein. A comprehensive review of the Cas protein family is presented in Haft et al. (2005) Computational Biology, PLoS Comput Biol 1(6): e60. doi:10.1371/journal.pcbi.0010060. As described therein, 41 CRISPR-associated (Cas) gene families are described, in addition to the four previously known gene families. It shows that CRISPR systems belong to different classes, with different repeat patterns, sets of genes, and species ranges. The number of Cas genes at a given CRISPR locus can vary between species.

As used herein, the term “guide RNA” refers to a synthetic fusion of two RNA molecules, a crRNA (CRISPR RNA) comprising a variable targeting domain, and a tracrRNA. The guide RNA may comprise a variable targeting domain of 12 to 30 nucleotide sequences and a RNA fragment that can interact with a Cas endonuclease.

The term “variable targeting domain” refers to a nucleotide sequence 5 -prime of the GUUUU sequence motif in the guide RNA, that is complementary to one strand of a double strand DNA target site in the genome of a plant cell, plant or seed. In one embodiment, the variable targeting domain is 12 to 30 nucleotides in length.

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151 153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids the parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., i Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

180,996 novel transcripts resulting in 47,457 novel proteins have been identified herein. Analysis of previously identified alternative transcripts (in U.S. application Ser. No. 14/628,469, filed Feb. 23, 2015) has shown that (1) newly identified transcripts may be truncated at their N or C terminus; (2) newly identified transcripts may be extensions at either terminus, thereby gaining new functional domains; (3) proteins encoded by the newly identified transcripts may have internal domains added, removed, or substituted without shifting their reading frames, in comparison to their most similar annotated transcripts; (4) proteins encoded by the newly identified transcripts could have less than 25% identity with their most similar annotated transcripts and could have a distinct function; and (5) newly identified transcripts may encode proteins that are identical to those generated by their most similar known transcripts but the transcripts possess different UTRs. In addition, the newly identified transcripts may result in new genes and isoforms which are potential miRNA targets or new genes and isoforms that have lost their target site.

Embodiments include isolated polynucleotides and polypeptides, recombinant DNA constructs useful for improving one or more agronomic characteristics in a plant, compositions (such as plants or seeds) comprising the recombinant DNA constructs, and methods utilizing the recombinant DNA constructs.

### Isolated Polynucleotides and Polypeptides:

Computational analysis of hundreds of RNA-seq libraries enabled the identification of novel transcripts in maize. Polynucleotides corresponding to the novel transcripts are provided herein, as are the polypeptides encoded by the polynucleotides. The polynucleotide sequences are represented by SEQ ID NOs:1-157,066 and 198,539-222,468, and the polypeptide sequences are represented by SEQ ID NOs:157,067-198,538 and 222,469-228,453.

An isolated polynucleotide comprising: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to any of SEQ ID NOs:157,067-198,538 and 222,469-228,453; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any recombinant DNA constructs disclosed herein.

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to any of SEQ ID NOs: 157,067-198,538 and 222,469-228,453.

An isolated polynucleotide comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to any of SEQ ID NOs:1-157,066 and 198,539-222,468; or (ii) a full complement of the nucleic acid sequence of (i). Any of the foregoing isolated polynucleotides may be utilized in any recombinant DNA constructs disclosed herein.

An isolated polynucleotide comprising a nucleotide sequence, wherein the nucleotide sequence is hybridizable under stringent conditions with a DNA molecule comprising the full complement of any of SEQ ID NOs:1-157,066 and 198,539-222,468.

An isolated polynucleotide comprising a nucleotide sequence, wherein the nucleotide sequence is derived from any of SEQ ID NOs:1-157,066 and 198,539-222,468 by alteration of one or more nucleotides by at least one method selected from the group consisting of: deletion, substitution, addition and insertion.

An isolated polynucleotide comprising a nucleotide sequence, wherein the nucleotide sequence corresponds to an allele of SEQ ID NOs:1-157,066 and 198,539-222,468.

Also of interest are fragments of the disclosed polynucleotides consisting of oligonucleotides of at least 15, preferably at least 16 or 17, more preferably at least 18 or 19, and even more preferably at least 20 or more, consecutive nucleotides. Such oligonucleotides are fragments of any of the larger polynucleotide sequences of SEQ ID NOs:1-157,066 and 198,539-222,468, and may find use, for example as probes and primers for detection of the polynucleotides disclosed herein.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N terminal and C terminal portions of the polypeptide molecule would also not be expected to alter the activity of the polypeptide. Each of the proposed modifications is well within the routine skill in the art, as is determination of retention of biological activity of the encoded products.

A protein disclosed herein may also be a protein which comprises an amino acid sequence comprising a deletion, substitution, insertion and/or addition of one or more amino acids in an amino acid sequence presented in any of SEQ ID NOs:157,067-198,538 and 222,469-228,453. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p.6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

A protein disclosed herein may also be a protein which is encoded by a nucleic acid comprising a nucleotide sequence comprising a deletion, substitution, insertion and/or addition of one or more nucleotides in the nucleotide sequence of any of SEQ ID NOs:1-157,066 and 198,539-222,468. Nucleotide deletion, substitution, insertion and/or addition may be accomplished by site-directed mutagenesis or other techniques as mentioned above.

A protein disclosed herein may also be a protein which is encoded by a nucleic acid comprising a nucleotide sequence hybridizable under stringent conditions with the complementary strand of the nucleotide sequence of any of SEQ ID NOs:1-157,066 and 198,539-222,468.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

### Recombinant DNA Constructs:

Recombinant DNA constructs comprising polynucleotides disclosed herein are also provided.

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to any of SEQ ID NOs:157,067-198,538 and 222,469-228,453; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to any of SEQ ID NOs:1-157,066 and 198,539-222,468; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide comprises (i) a nucleic acid sequence that is transcribed into an RNA molecule that suppresses the level of an endogenous polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to any of SEQ ID NOs:157,067-198,538 and 222,469-228,453.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N terminal and C terminal portions of the polypeptide molecule would also not be expected to alter the activity of the polypeptide. Each of the proposed modifications is well within the routine skill in the art, as is determination of retention of biological activity of the encoded products.

The recombinant DNA construct may be a suppression DNA construct and may comprise a cosuppression construct, antisense construct, viral-suppression construct, hairpin suppression construct, stem-loop suppression construct, double-stranded RNA-producing construct, RNAi construct, or small RNA construct (e.g., an sRNA construct or an miRNA construct).

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

A suppression DNA construct may comprise 100, 200, 300, 400, 500, 600, 700, 800, 900 or 1000 contiguous nucleotides of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as sRNA (short interfering RNA) constructs and miRNA (microRNA) constructs.

Suppression of gene expression may also be achieved by use of artificial miRNA precursors, ribozyme constructs and gene disruption. A modified plant miRNA precursor may be used, wherein the precursor has been modified to replace the miRNA encoding region with a sequence designed to produce a miRNA directed to the nucleotide sequence of interest. Gene disruption may be achieved by use of transposable elements or by use of chemical agents that cause site-specific mutations.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on August 20, 1998).

RNA interference refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

The terms “miRNA-star sequence” and “miRNA*sequence” are used interchangeably herein and they refer to a sequence in the miRNA precursor that is highly complementary to the miRNA sequence. The miRNA and miRNA*sequences form part of the stem region of the miRNA precursor hairpin structure.

In one embodiment, there is provided a method for the suppression of a target sequence comprising introducing into a cell a nucleic acid construct encoding a miRNA substantially complementary to the target. In some embodiments the miRNA comprises about 19, 20, 21, 22, 23, 24 or 25 nucleotides. In some embodiments the miRNA comprises 21 nucleotides. In some embodiments the nucleic acid construct encodes the miRNA. In some embodiments the nucleic acid construct encodes a polynucleotide precursor which may form a double-stranded RNA, or hairpin structure comprising the miRNA.

In some embodiments, the nucleic acid construct comprises a modified endogenous plant miRNA precursor, wherein the precursor has been modified to replace the endogenous miRNA encoding region with a sequence designed to produce a miRNA directed to the target sequence. The plant miRNA precursor may be full-length of may comprise a fragment of the full-length precursor. In some embodiments, the endogenous plant miRNA precursor is from a dicot or a monocot. In some embodiments the endogenous miRNA precursor is from Arabidopsis, tomato, maize, soybean, sunflower, sorghum, canola, wheat, alfalfa, cotton, rice, barley, millet, sugar cane or switchgrass.

In some embodiments, the miRNA template, (i.e. the polynucleotide encoding the miRNA), and thereby the miRNA, may comprise some mismatches relative to the target sequence. In some embodiments the miRNA template has >1 nucleotide mismatch as compared to the target sequence, for example, the miRNA template can have 1, 2, 3, 4, 5, or more mismatches as compared to the target sequence. This degree of mismatch may also be described by determining the percent identity of the miRNA template to the complement of the target sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the target sequence.

In some embodiments, the miRNA template, (i.e. the polynucleotide encoding the miRNA) and thereby the miRNA, may comprise some mismatches relative to the miRNA-star sequence. In some embodiments the miRNA template has >1 nucleotide mismatch as compared to the miRNA-star sequence, for example, the miRNA template can have 1, 2, 3, 4, 5, or more mismatches as compared to the miRNA-star sequence. This degree of mismatch may also be described by determining the percent identity of the miRNA template to the complement of the miRNA-star sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the miRNA-star sequence.

In some embodiments, the nucleic acid constructs express one or more guide RNAs, wherein a guide RNA targets a genomic sequence that encodes a polypeptide having any of the amino acid sequences set forth in SEQ ID NOs: 157,067-198,538 and 222,469-228,453.

### Regulatory Sequences:

A recombinant DNA construct as disclosed herein may comprise at least one regulatory sequence.

A regulatory sequence may be a promoter.

A number of promoters can be used in recombinant DNA constructs disclosed herein. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance drought tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods disclosed herein, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods disclosed herein which causes the desired temporal and spatial expression.

Promoters which are seed or embryo-specific and may include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters that can be used in the context of the current disclosure may include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and CimI which is specific to the nucleus of developing maize kernels. CimI transcript is detected 4 to 5 days before pollination to 6 to 8 DAP. Other useful promoters include any promoter which can be derived from a gene whose expression is maternally associated with developing female florets.

Additional promoters for regulating the expression of the nucleotide sequences disclosed herein may include stalk-specific promoters such as the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

In one embodiment the at least one regulatory element may be an endogenous promoter operably linked to at least one enhancer element; e.g., a 35S, nos or ocs enhancer element.

Promoters for use herein may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664),

Recombinant DNA constructs as disclosed herein may also include other regulatory sequences, including but not limited to, translation leader sequences, introns, and polyadenylation recognition sequences. In another embodiment, a recombinant DNA construct disclosed herein may further comprise an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

Any plant can be selected for the identification of regulatory sequences and genes to be used in recombinant DNA constructs, other compositions (e.g. transgenic plants, seeds and cells), and methods as disclosed herein. Examples of suitable plants for the isolation of genes and regulatory sequences and for compositions and methods disclosed herein may include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yams, and zucchini.

### Compositions:

A composition as disclosed herein may include a transgenic microorganism, cell, plant, or seed comprising the recombinant DNA construct. The cell may be eukaryotic, e.g., a yeast, insect or plant cell, or prokaryotic, e.g., a bacterial cell.

A composition disclosed herein may be a plant comprising in its genome any of the polynucleotide sequences and/or recombinant DNA constructs disclosed herein. Compositions also include any progeny of the plant, and any seed obtained from the plant or its progeny, wherein the progeny or seed comprises within its genome the recombinant DNA construct. Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct. These seeds can be grown to produce plants that would exhibit an improved agronomic characteristic, or used in a breeding program to produce hybrid seed, which can be grown to produce plants that would exhibit such an improved agronomic characteristic. The seeds may be maize seeds.

The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass. The plant may be a hybrid plant or an inbred plant.

The recombinant DNA construct may be stably integrated into the genome of the plant.

Any of the polynucleotides described herein may be stably integrated into the genome of a plant using genome editing. Thus, a plant comprising a heterologous regulatory element operably linked to any of the polynucleotide sequences presented herein (SEQ ID NOs:1-157,066 and 198,539-222,468) is also provided.

In any of the embodiments described herein, the recombinant DNA construct may comprise at least a promoter functional in a plant as a regulatory sequence.

In any of the embodiments described herein, the at least one agronomic characteristic may be selected from the group consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

One of ordinary skill in the art would readily recognize a suitable control or reference plant to be utilized when assessing or measuring an agronomic characteristic or phenotype of a transgenic plant in any embodiment described herein in which a control plant is utilized (e.g., compositions or methods as described herein). For example, by way of non-limiting illustrations:
1. Progeny of a transformed plant which is hemizygous with respect to a recombinant DNA construct, such that the progeny are segregating into plants either comprising or not comprising the recombinant DNA construct: the progeny comprising the recombinant DNA construct would be typically measured relative to the progeny not comprising the recombinant DNA construct (i.e., the progeny not comprising the recombinant DNA construct is the control or reference plant).
2. Introgression of a recombinant DNA construct into an inbred line, such as in maize, or into a variety, such as in soybean: the introgressed line would typically be measured relative to the parent inbred or variety line (i.e., the parent inbred or variety line is the control or reference plant).
3. Two hybrid lines, where the first hybrid line is produced from two parent inbred lines, and the second hybrid line is produced from the same two parent inbred lines except that one of the parent inbred lines contains a recombinant DNA construct: the second hybrid line would typically be measured relative to the first hybrid line (i.e., the first hybrid line is the control or reference plant).
4. A plant comprising a recombinant DNA construct: the plant may be assessed or measured relative to a control plant not comprising the recombinant DNA construct but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

### Methods:

Polynucleotides presented herein can be used to improve agronomic characteristics by providing for enhanced protein activity in a transgenic organism, preferably a transgenic plant, although in some cases, improved properties are obtained by providing for reduced protein activity in a transgenic plant. Reduced protein activity and enhanced protein activity are measured by reference to a wild type cell or organism, and can be determined by direct or indirect measurement. Direct measurement of protein activity might include an analytical assay for the protein, per se, or enzymatic product of protein activity. Indirect assay might include measurement of a property affected by the protein. Enhanced protein activity can be achieved in a number of ways, for example by overproduction of mRNA encoding the protein or by gene shuffling. One skilled in the art will know methods to achieve overproduction of mRNA, for example by providing increased copies of the native gene or by introducing a construct having a heterologous promoter linked to the gene into a target cell or organism. Reduced protein activity can be achieved by a variety of mechanisms including antisense, mutation or knockout. Antisense RNA will reduce the level of expressed protein resulting in reduced protein activity as compared to wild type activity levels. A mutation in the gene encoding a protein may reduce the level of expressed protein and/or interfere with the function of expressed protein to cause reduced protein activity.

The polypeptides may be involved in one or more important biological properties in plants. Such polypeptides may be produced in transgenic plants to provide plants having improved agronomic characteristics. In some cases, decreased expression of such polypeptides may be desired, such decreased expression being obtained by use of the polynucleotide sequences provided herein, for example in antisense or cosuppression methods.

Methods include but are not limited to methods for improving at least one agronomic characteristic in a plant, methods for determining an alteration of an agronomic characteristic in a plant, and methods for producing seed. The plant may be a monocotyledonous or dicotyledonous plant, for example, a maize or soybean plant. The plant may also be sunflower, sorghum, canola, wheat, alfalfa, cotton, rice, barley, millet, sugar cane or sorghum. The seed may be a maize or soybean seed, for example, a maize hybrid seed or maize inbred seed.

A method for transforming a cell (or microorganism) comprising transforming a cell (or microorganism) with any of the isolated polynucleotides or recombinant DNA constructs disclosed herein is provided. The cell (or microorganism) transformed by this method is also included. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes.

A method for producing a transgenic plant comprising transforming a plant cell with any of the isolated polynucleotides or recombinant DNA constructs disclosed herein and regenerating a transgenic plant from the transformed plant cell is also provided. A transgenic plant produced by this method, which may have at least one improved agronomic characteristic, and transgenic seed obtained from this transgenic plant are also provided. The transgenic plant obtained by this method may be used in other methods disclosed herein.

A method for isolating a polypeptide disclosed herein from a cell or culture medium of the cell, wherein the cell comprises a recombinant DNA construct comprising a polynucleotide disclosed herein operably linked to at least one regulatory sequence, and wherein the transformed host cell is grown under conditions that are suitable for expression of the recombinant DNA construct is provided.

A method of altering the level of expression of a polypeptide disclosed herein in a host cell is provided herein. The method comprises: (a) transforming a host cell with a recombinant DNA construct disclosed herein; and (b) growing the transformed host cell under conditions that are suitable for expression of the recombinant DNA construct wherein expression of the recombinant DNA construct results in production of altered levels of the polypeptide in the transformed host cell.

A method of selecting for (or identifying) an alteration of an agronomic characteristic in a plant, comprising (a) obtaining a transgenic plant, wherein the transgenic plant comprises in its genome a recombinant DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to any of the SEQ ID NOs:157,067-198,538 and 222,469-228,453; (b) obtaining a progeny plant derived from said transgenic plant, wherein the progeny plant comprises in its genome the recombinant DNA construct; and (c) selecting (or identifying) the progeny plant that exhibits an alteration in at least one agronomic characteristic when compared to a control plant not comprising the recombinant DNA construct.

In another embodiment, a method of selecting for (or identifying) an alteration of at least one agronomic characteristic in a plant, comprising: (a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to any of SEQ ID NOs:157,067-198,538 and 222,469-228,453, wherein the transgenic plant comprises in its genome the recombinant DNA construct; (b) growing the transgenic plant of part (a) under conditions wherein the polynucleotide is expressed; and (c) selecting (or identifying) the transgenic plant of part (b) that exhibits an alteration of at least one agronomic characteristic when compared to a control plant not comprising the recombinant DNA construct.

A method of selecting for (or identifying) an alteration of an agronomic characteristic in a plant, comprising (a) obtaining a transgenic plant, wherein the transgenic plant comprises in its genome a recombinant DNA construct comprising a polynucleotide operably linked to at least one regulatory element, wherein said polynucleotide comprises a nucleotide sequence, wherein the nucleotide sequence is: (i) hybridizable under stringent conditions with a DNA molecule comprising the full complement of any of SEQ ID NOs:1-157,066 and 198,539-222,468; or (ii) derived from any of SEQ ID NOs:1-157,066 and 198,539-222,468 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) selecting (or identifying) the progeny plant that exhibits an alteration in at least one agronomic characteristic when compared to a control plant not comprising the recombinant DNA construct.

A method of selecting for (or identifying) an alteration of an agronomic characteristic in a plant, comprising (a) obtaining a transgenic plant, wherein the transgenic plant comprises in its genome a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to any of SEQ ID NOs:157,067-198,538 and 222,469-228,453, or (ii) a full complement of the nucleic acid sequence of (i); (b) obtaining a progeny plant derived from said transgenic plant, wherein the progeny plant comprises in its genome the suppression DNA construct; and (c) selecting (or identifying) the progeny plant that exhibits an alteration in at least one agronomic characteristic when compared to a control plant not comprising the suppression DNA construct. A method of producing seed comprising any of the preceding methods, and further comprising obtaining seeds from said progeny plant, wherein said seeds comprise in their genome said recombinant DNA construct (or suppression DNA construct).

A method for enhancing expression of a transgene in a plant are provided in which a nucleotide sequence of a transgene or an amino acid sequence of a transgene are obtained; the sequences are compared to a collection of nucleotide sequences of alternatively spliced isoforms or to a collection of amino acid sequences encoded by the alternatively spliced isoforms; one or more alternatively spliced isoform sequences corresponding to a transgene are selected; and the one or more alternatively spliced isoform sequences in the plant are expressed, thereby enhancing expression of the transgene. The selected isoform sequence may be expressed under its native promoter or a constitutive or tissue-preferred promoter.

A method for introducing any of the polynucleotides disclosed herein into a target site in the genome of a plant cell is also provided. The method comprises (a) introducing into a plant cell one recombinant DNA construct capable of expressing a guide RNA and another recombinant DNA construct capable of expressing a Cas endonuclease, wherein said guide RNA and Cas endonuclease are capable of forming a complex that enables the Cas endonuclease to introduce a double strand break at said target site; (b) contacting the plant cell with a donor DNA comprising a polynucleotide of interest, wherein said polynucleotide of interest is any of the polynucleotides disclosed herein; and (c) identifying at least one plant cell that has the polynucleotide of Interest integrated into the target site.

A method of editing a genome to alter splice sites is also provided herein. The method may involve introducing one or more heterologous splice sites or eliminating one or more splice sites. The method includes identifying one or more alternatively spliced isoforms; determining one or more splice sites in the genomic region for the alternatively spliced isoforms; and introducing a splice site in the genomic loci that lacks the one or more splice sites or changing one or more nucleotides in a preexisting splice site to render the preexisting splice site non-functional. The alternatively spliced isoforms may be selected from the group consisting of SEQ ID NOs: 1-157,066 and 198,539-222,468.

Other methods to modify or alter the host endogenous genomic DNA are also available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” or engineered endonucleases such as meganucleases produced to modify plant genomes (see e.g., WO 2009/114321; Gao et al. (2010) Plant Journal 1:176-187). Another site-directed engineering is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326(5959): 1509-12.

In any of the preceding methods or any other embodiments of methods disclosed herein, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods disclosed herein, said regenerating step may comprise the following: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods disclosed herein, the at least one agronomic characteristic may be selected from the group consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress. The agronomic characteristic may be abiotic stress tolerance, such as for example, tolerance to nutrient deprivation (e.g. nitrogen) or to drought.

In any of the preceding methods or any other embodiments of methods disclosed herein, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide disclosed herein.

The introduction of recombinant DNA constructs disclosed herein into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant disclosed herein that contains a desired polypeptide can be cultivated using methods well known to one skilled in the art.

### Other Methods of Interest

A method of marker assisted selection of a maize plant is also provided herein. The method involves: analyzing for expression of one or more transcripts selected from a group consisting of nucleotide sequences, wherein the nucleotide sequences encode alternatively spliced isoforms; correlating one or more transcripts with an improved agronomic characteristic; and selecting for the improved agronomic characteristic in a maize plant by assaying one or more markers that detect the one or more transcripts associated with the improved agronomic characteristic. The expression analysis may be performed with a plurality of isoform-specific probes derived from the group consisting of sequences SEQ ID NOs:1-157,066 and 198,539-222,468.

A method of identifying alternatively spliced isoforms of one or more genes involved in an agronomic trait are also provided in which a plurality of transcripts that are expressed under an abiotic stress condition are sequenced and the sequenced transcripts are compared to transcript sequences that are expressed in a non-stressed condition. Genes with splicing patterns that differ between the abiotic stress condition and non-stressed condition are then detected.

A method for comparing a plurality of spliced isoforms among two or more plant populations, comprising: (a) accessing, by a computer system, a database of genetic information comprising spliced isoform sequences obtained from a plurality of plant tissues; (b) categorizing, by a computer system, the data in the database into a plurality of groups of spliced isoforms, such that one or more spliced isoforms for a particular gene are in the same group, and each group represents a different set of spliced isoforms; and (c) inputting data into a computer system, the data comprising sequences of one or more transcripts obtained from the two or more plant populations, is also provided. The plant populations may comprise inbred populations. The database may further comprise QTL information associated with one or more spliced isoforms.

### Computer Systems and Programs

Computer systems comprising: a relational database having records containing a) information about one or more sequences of spliced isoforms represented by SEQ ID NOs: 1-157,066 and 198,539-222,468 or amino acid sequences of 157,067-198,538 and 222,469-228,453; b) information identifying known SNPs or QTLs known to be associated with one or more traits of interest; and c) a user interface allowing a user to access the information contained in the records, are also provided.

Computer programs comprising: a computer-usable medium having computer-readable program code embodied thereon relating to generating a relational database having records containing a) information about one or more sequences of spliced isoforms represented by SEQ ID NOS: 1-157,066 and 198,539-222,468 or amino acid sequences of 157,067-198,538 and 222,469-228,453; b) information identifying known SNPs or QTLs known to be associated with one or more traits of interest; and c) a user interface allowing a user to access the information contained in the records, are also provided.

### EXAMPLES

The present invention is further illustrated in the following Examples, in which parts and percentages are by weight and degrees are Celsius, unless otherwise stated. It should be understood that the Examples, while indicating embodiments of the invention, are given by way of illustration only. From the above discussion and the Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Prediction of Novel Transcripts

In order to discover and map novel transcripts in maize, 94 paired-end RNA seq libraries were constructed from 5 week old leaves of three B73, three Mo17 and 88 intermated B73×Mo17 (IBM) Syn10 double haploid (DH) lines. The IBM mapping population was originally created through ten generations of B73 and Mo17 intermating, followed by double haploid generation and resulted in a population containing highly recombinant fixed alleles (Hussain et al. 2007. Journal of Plant Registrations 1:81). More than six billion genome-matched reads were obtained (Table 1).

Transcript discovery was also augmented by the inclusion of 142 publically available B73 RNA seq libraries originating from 14 different tissue types, totaling over two billion genome-matched reads (Table 2). All libraries were genome matched using Tophat2 (Kim et al. 2013. Genome Biology 14(4):R36), followed by novel isoform discovery using the Cufflinks pipeline (Trapnell et al. 2010. Nature Biotech 28(5):511-515) with a working set of 137,000 annotated public maize (Gramene release 5a).

TABLE 1
Summary statistics for B73, Mo17, and IBM RNA seq libraries

| Description | Genotype | Libraries | Total Reads | Genome Matched |
|---|---|---|---|---|
| B73 | B73 | 3 | 252,961,366 | 249,428,288 |
| Mo17 | Mo17 | 3 | 405,071,583 | 393,962,382 |
| IBM | IBM | 88 | 5,795,253,356 | 5,599,170,515 |

TABLE 2
Summary statistics for RNA seq libraries

| Description | Genotype | Libraries | Total Reads | Genome Matched |
|---|---|---|---|---|
| Anther | B73 | 1 | 38,074,756 | 36,554,492 |
| Ear | B73 | 4 | 104,293,259 | 98,987,393 |
| Embryo | B73 | 7 | 60,710,425 | 55,861,189 |
| Endosperm | B73 | 13 | 144,540,885 | 131,347,944 |
| Leaf | B73 | 42 | 664,025,044 | 618,671,115 |
| Ovule | B73 | 1 | 36,964,181 | 35,379,281 |
| Pollen | B73 | 1 | 38,623,695 | 37,342,145 |
| Root | B73 | 18 | 296,713,582 | 272,807,740 |
| SAM | B73 | 10 | 148,544,984 | 135,325,790 |
| Seed | B73 | 20 | 346,866,162 | 320,235,834 |
| Seedling | B73 | 2 | 23,661,408 | 22,675,374 |
| Shoot | B73 | 14 | 136,391,616 | 121,490,509 |
| Silk | B73 | 1 | 24,398,322 | 23,372,552 |
| Tassel | B73 | 8 | 175,790,705 | 166,472,788 |

Isoform prediction from public data and the IBM population were initially carried out as two separate analyses, yet generated a novel isoform set with a high degree of overlap. The entire content of U.S. application number Ser. No. 14/628,469, filed Feb. 23, 2015 is hereby incorporated by reference.

### Example 2

### Comparison of Novel Transcripts with an Artificial Randomly Generated Set

In order to assess the quality and ideal abundance cutoff for novel isoforms, a set of artificial isoforms based on known transcripts was created. One artificial isoform was randomly generated for each annotated transcript by modification of the known transcript based on alternative splicing categories: intron retention, exon skipping, alternative donor, alternative acceptor and alternative position. The 137,000 artificial transcripts each differed from a known transcript by one random splicing modification, making them an ideal set to compare against.

To determine an abundance cutoff for novel isoforms, known and randomly generated transcripts were quantified using Cuffdiff (Roberts et al. 2011. Bioinformatics 27(17):2325-2329) in the fourteen public libraries, as well as B73, Mo17 parents and IBM DH lines. Cutoffs ranging from 0.01 to 10 FPKM (Fragments Per Kilobase of transcript per Million mapped reads) were applied and the fraction of transcripts having expression above each cutoff in at least one tissue was determined. At an extremely low expression cutoff of 0.01 FPKM, 72% of known transcripts were expressed in at least one tissue, while 57% of artificially generated transcripts were similarly expressed. Taking 0.01 as the basal expression level, the loss of known transcripts as the abundance cutoff increased (false negatives) was then plotted against the loss of artificial transcripts (i.e. false positives). To increase the number of novel isoforms identified, a relaxed filter of 0.5 FPKM was utilized, resulting in the cDNA sequences represented by SEQ ID NOs:1-157,066. The novel proteins encoded by the newly identified isoforms/genes using the 0.5 FPKM filter are represented by SEQ ID NOs:157,067-198,538. Table 3 provides the SEQ ID NO: for each isoform identified using 0.5 FPKM as the expression cutoff.

### Example 3

### mRNA Seq Library Preparation and Sequencing

In addition to the experiments shown in Examples 1 and 2, total RNA was isolated from frozen maize tissues with Qiagen RNeasy kit for total RNA isolation (Qiagen, Valencia, Calif. USA). Libraries from total RNA were then prepared using the TruSeq mRNA-Seq kit and protocol from Illumina, Inc. (San Diego, Calif., USA) and sequenced on the Illumina HiSeq 2500 system with Illumina TruSeq SBS v3 reagents. On average, 18 million reads were generated from each library (Table 4). Resulting sequences were trimmed based on quality scores (Phred score >13) and mapped to the maize B73 reference genome sequence V2 and maize working gene set V5a with Tophat2 version 2.0.14 (Kim et al., 2013 supra) using several modifications from default parameters; maximum intron size: 100,000, minimum intron size: 20, up to two mismatches allowed. Reads which aligned to multiple locations were assigned heuristically based on the abundance of surrounding regions (Kim et al., 2013 supra). Libraries with less than 5,000,000 genome-matched reads (one biological replicate of well-watered R1 tassel, and one biological replicate of drought-stressed R1 tassel) were excluded from down later downstream analysis.

TABLE 4
Summary statistics for RNA seq libraries

| Description | Condition | Stages | Libraries | Total Reads | Genome Matched | Uniquely Mapped | Percent Mapped |
|---|---|---|---|---|---|---|---|
| Ear | watered | 4 | 16 | 286,248,214 | 261,827,251 | 233,864,244 | 91% |
| Tassel | watered | 4 | 16 | 215,381,271 | 205,082,097 | 180,285,669 | 95% |
| Leaf | watered | 4 | 16 | 230,210,634 | 204,178,390 | 183,473,272 | 89% |
| Ear | drought | 4 | 16 | 299,932,575 | 256,161,636 | 229,619,752 | 86% |
| Tassel | drought | 4 | 14 | 207,741,601 | 197,232,548 | 173,040,626 | 95% |
| Leaf | drought | 4 | 16 | 241,874,221 | 224,097,862 | 202,057,363 | 93% |
| Seed | watered | 21 | 28 | 381,174,028 | 336,649,861 | 309,166,899 | 88% |
| Endosperm | watered | 17 | 21 | 332,421,896 | 300,859,239 | 267,102,578 | 91% |
| Embryo | watered | 15 | 16 | 217,398,766 | 197,532,915 | 189,422,320 | 91% |

### Example 4

### Computational Prediction of Novel Isoforms

Genome-matched reads from each library then were assembled with Cufflinks version 2.1.1 (Trapnell et al., 2010 supra) using several modifications from default parameters; maximum intron size: 100,000, minimum intron size: 20. Cuffmerge version 2.1.1 (Roberts et al., 2011 supra) was then used to merge individual transcript assemblies into a single transcript set. Annotation of novel junctions required at least 10 reads spanning them and any new transcripts needed to represent at least 10% of the total gene abundance in at least one library. Known and novel transcripts were quantified in each tissue and genotype with Cuffnorm version 2.1.1 (Roberts et al., 2011 supra) using default parameters. Novel transcripts with expression less than 1.3 FPKM in all tissues and stages were filtered out. Table 5 provides the SEQ ID NO: for each novel transcript identified (SEQ ID NOs:198,539-222,468 represent the cDNAs; SEQ ID NOs:222,469-228,453 represent the polypeptides).

What is claimed is:

1. A recombinant DNA construct comprising a polynucleotide operably linked to at least one regulatory sequence wherein said polynucleotide comprises:
a. a nucleic acid sequence of at least 95% sequence identity, based on the Clustal V method of alignment, when compared to any of SEQ ID NOs:1-157,066 and 198,539-222,468;
b. a nucleic acid sequence encoding an amino acid sequence of at least 95% sequence identity, based on the Clustal V method of alignment, when compared to any of SEQ ID NOs:157,067-198,538 and 222,469-228,453; or
c. a nucleic acid sequence that is transcribed into an RNA molecule that suppresses the level of an endogenous polypeptide having an amino acid sequence of at least 95% sequence identity, based on the Clustal V method of alignment, when compared to any of SEQ ID NOs:157,067-198,538 and 222,469-228,453.

2. The recombinant DNA construct of claim 1, wherein said at least one regulatory sequence is a promoter functional in a plant cell.

3. A transgenic plant cell comprising the recombinant DNA construct of claim 1.

4. A transgenic plant comprising the transgenic plant cell of claim 3.

5. The transgenic plant of claim 4, wherein said transgenic plant is selected from the group consisting of: Arabidopsis, maize, soybean, sunflower, sorghum, canola, wheat, alfalfa, cotton, rice, barley, millet, sugar cane and switchgrass.

6. Transgenic seed produced from the transgenic plant of claim 4.

7. A method of producing a transgenic plant having an improved agronomic characteristic, wherein said method comprises:
a. transforming a plant cell with the recombinant DNA construct of claim 1; and
b. regenerating a plant from the transformed plant cell.

8. A method for introducing a polynucleotide of Interest into a target site in the genome of a plant cell, the method comprising:
a. introducing into a plant cell a first recombinant DNA construct capable of expressing a guide RNA and a second recombinant DNA construct capable of expressing a Cas endonuclease, wherein said guide RNA and Cas endonuclease are capable of forming a complex that enables the Cas endonuclease to introduce a double strand break at said target site;
b. contacting the plant cell of (a) with a donor DNA comprising a polynucleotide of interest, wherein said polynucleotide of interest is:
i. a nucleic acid sequence of at least 95% sequence identity, based on the Clustal V method of alignment, when compared to any of SEQ ID NOs:1-157,066 and 198,539-222,468; or
ii. a nucleic acid sequence encoding an amino acid sequence of at least 95% sequence identity, based on the Clustal V method of alignment, when compared to any of SEQ ID NOs:157,067-198,538 and 222,469-228,453; and
c. identifying at least one plant cell from (b) comprising in its genome the polynucleotide of Interest integrated at said target site.

9. A method of marker assisted selection of a maize plant, the method comprising:
a. analyzing for expression of one or more transcripts selected from a group consisting of nucleotide sequences, wherein the nucleotide sequences encode alternatively spliced isoforms;
b. correlating one or more transcripts with an improved agronomic characteristic; and
c. selecting for the improved agronomic characteristic in a maize plant by assaying one or more markers that detect the one or more transcripts associated with the improved agronomic characteristic.

10. The method of claim 9, wherein the expression analysis is performed with a plurality of isoform-specific probes derived from the group consisting of sequences SEQ ID NOs:1-157,066 and 198,539-222,468.
